(19) United States
(12) Patent Application Publication
(10) Pub. No.: US 2023/0277451 A1
(43) Pub. Date: Sep. 7, 2023

(54) A PHARMACEUTICAL FORMULATION FOR PRESSURISED METERED DOSE INHALER

(71) Applicant: CHIESI FARMACEUTICI S.P.A., Parma (IT)

(72) Inventors: Enrico ZAMBELLI, Parma (IT); Sauro BONELLI, Parma (IT); Diego COPELLI, Parma (IT); Massimiliano DAGLI ALBERI, Parma (IT); Francesca USBERTI, Parma (IT)

(73) Assignee: CHIESI FARMACEUTICI S.P.A., Parma (IT)

(21) Appl. No.: 18/030,153

(22) PCT Filed: Oct. 8, 2021
(86) PCT No.: PCT/EP2021/077827

§ 371 Date: Apr. 4, 2023

(30) Foreign Application Priority Data
Oct. 9, 2020 (EP) 20201060.9

(51) Int. Cl. A61K 9/00; A61K 31/167; A61K 47/02; A61K 31/40; A61K 31/573

(52) U.S. Cl. CPC A61K 9/008; A61K 31/167; A61K 47/02; A61K 31/40; A61K 31/573

(57) ABSTRACT

The present invention generally relates to pharmaceutical composition comprising a LABA agent, optionally in combination with other active ingredients, a mixture of at least two inorganic acids, a propellant and a co-solvent. The invention also provides a pharmaceutical composition for the treatment of respiratory diseases, such as asthma and COPD.

### FIELD OF THE INVENTION

The present invention generally relates to a pharmaceutical composition comprising a LABA agent, a mixture of at least two inorganic acids, a propellant and a co-solvent; the invention further relates to the use of such pharmaceutical compositions in the treatment and prevention of respiratory diseases.

### BACKGROUND OF THE INVENTION

Pressurized metered dose inhalers (pMDIs) are well known devices for administering pharmaceutical products to the respiratory tract by inhalation. A pMDI device typically presents a medical-containing canister (or a “can” as herein referred to), and an actuator housing having a mouthpiece. The can is usually crimped with a metered valve assembly. Depending on the active ingredients and on additional components such as excipients, acids and similar, a final pMDI formulation may be in the form of a solution or a suspension. As known in the art, solution is generally intended as substantially lacking precipitates or particles, while suspension typically refers to formulation having some undissolved material or precipitates. pMDI devices may use a propellant to expel droplets containing the pharmaceutical products to the respiratory tract as an aerosol.

Glycopyrronium bromide (also known as glycopyrrolate), classified among the long-acting muscarinic antagonists (LAMA's), is a particularly efficacious bronchodilator in the treatment of respiratory diseases when in combination with LABA agents and corticosteroids.

Aerosol inhalation compositions suitable for a pMDI device comprising formoterol in combination with glycopyrronium bromide have been described in literatures.

WO 2011/076842 describes a pharmaceutical composition comprising glycopyrronium bromide dissolved in HFA propellant and a co-solvent, containing an amount of 1M hydrochloric acid (HCl) wherein the formulation shows a good stability profile.

WO 2011/076843 describes a stabilized pharmaceutical composition comprising formoterol, glycopyrronium bromide dissolved in HFA propellant and a co-solvent wherein the formulation contains an amount of 1M HCl comprised in the range 0.1-0.3 μg/μl.

WO 2015/101576 describes a pMDI device particularly suitable for the use with a formoterol, beclometasone dipropionate and glycopyrronium bromide solution, contained in a FEP coated can. As therein disclosed, the formulation contained in a FEP coated can is endowed with an improved stability and reduced amount of degradation products, mainly with regards to the N-(3-bromo)-[2-hydroxy-5-[1-hydroxy-2-[1-(4-methoxyphenyl)propan-2-ylamino]ethyl]phenyl]formamide.

The chemical stability of the active pharmaceutical ingredients (APIs) contained in the pharmaceutical compositions is particularly desirable in order to obtain formulations suitable for the market, and to ensure the delivery of a constant dose of active ingredients per actuation.

Although the above-mentioned prior art provides effective formulations and devices technical arrangements, there is still the need to find an alternative aerosol formulation comprising a LABA agent particularly in combination with a LAMA agent and a corticosteroid, that is stable over an extended product lifetime, with the possibility to use commercially available cans, such as made of aluminium or stainless steel.

We have surprisingly found that the inclusion of a mixture of inorganic acids in a formulation comprising a LABA agent, optionally in combination with a LAMA agent and/or a corticosteroids substantially avoids the degradation of said active ingredients, thus maintaining the formulation stable over an extended period, and also exploiting an improvement in the stability profile of the formulation when suitable conditions are achieved, even when the formulation is contained in an aluminum canister.

Advantageously, said aerosol formulations comprising a mixture of inorganic acids as herein described, when formulated in a propellant, in the presence of a co-solvent can be usable in a pMDI device, particularly for the treatment of respiratory diseases, such as asthma and/or COPD, with excellent aerosolizing performances.

### SUMMARY OF THE INVENTION

In one aspect, the present invention refers to a pharmaceutical composition comprising a LABA agent, a co-solvent, a propellant and a mixture of at least two inorganic acids, preferably HCl and H₃PO₄.

Particularly, the invention refers to such a formulation, also comprising a LAMA agent and a corticosteroid agent.

In a further aspect, the invention refers to the use of said pharmaceutical composition comprising a LABA agent, a co-solvent, a propellant and a mixture of at least two inorganic acids for use as a medicament.

In a furthers aspect, the invention further relates to the use of a pharmaceutical composition comprising a LABA agent, a co-solvent, a propellant and a mixture of at least two inorganic acids, for the treatment and/or prophylaxis of respiratory disorders, in particular asthma and COPD.

### DETAILED DESCRIPTION OF THE INVENTION

Unless otherwise defined, all technical and scientific terms used herein have the same meanings as commonly understood by the skilled in the art.

The “molar ratio” between formoterol or a salt thereof or a solvate of said salt and the acid is calculated considering the number of moles of formoterol or a salt thereof or a solvate of said salt within the formulation and number of moles of the selected acid in the formulation.

Unless otherwise indicated the term “LABA” or “LABA agent” includes in its meaning a long acting beta 2 agonist, as known in the art, such as formoterol fumarate, arformoterol, or fenoterol.

Unless otherwise provided, the term “formoterol fumarate” or “FF” refers to (R,R)-(±)formoterol fumarate or dihydrate thereof.

Unless otherwise indicated the term “LAMA” or “LAMA agent” includes in its meaning a long acting muscarinic receptor antagonist, as known in the art, such a glycopyrronium, methscopolamine, ipratropium.

Glycopyrronium bromide, chemically defined as 3-[(cyclopentylhydroxyphenylacetyl)oxy]-1,1-dimethylpyrrolidinium bromide, has two chiral centres corresponding to four potential different stereoisomers with configurations (3R,2′R)-, (3S,2′R)-, (3R,2′S)- and (3S,2′S)-. Glycopyrronium bromide in the form of any of these pure enantiomers or diastereomers or any combination thereof may be used in practicing the present invention.

Unless otherwise indicated the term “glycopyrronium bromide” refers to (3 S,2′R),(3R,2′S)-3-[(cyclopentylhydroxyphenylacetyl)oxy]-1,1-dimethylpyrrolidinium bromide racemic mixture known also as glycopyrrolate (USAN name).

The term “% w/w” means the weight percentage of the component in respect to the total weight of the formulation.

The term “% w/v” means the weight percentage of the component in respect to the total volume of the formulation.

Regarding the term “apparent pH” as herein intended, it is noticed that the calculation of the pH is generally characteristic of aqueous liquid, e.g. where water is the dominant component. In relatively aprotic solvents (such as the propellants used in the present invention, e.g. an HFA or HFO system) protons are non-hydrated and their activity coefficients can differ from those in aqueous solution. Although the Nerst equation (describing potential of electrochemical cell as a function of concentrations of ions taking part in the reaction) with respect to electromagnetic field (EMF) applies and the pH-meter glass electrode system will generate a variable milli-volt output according to proton concentration and vehicle polarity, the pH meter reading represents the “apparent pH” according to the present invention. In this direction, the apparent pH according to the invention can be measured by technologies known in the art, as e.g. indicated in “Correlation between Apparent pH and Acid or Base Concentration in ASTM Medium” Orest Popovych, Analytical Chemistry 1964, 36,4,878-882; Analytical Standard Test Method (ASTM) D6423-19 “Standard Test Method for Determination of pH of Denatured Fuel Ethanol and Ethanol Fuel Blends”.

As above mentioned, the present invention unexpectedly shows that the inclusion of a mixture of inorganic acids in the formulation comprising a LABA agent, optionally in combination with a LAMA agent and/or a corticosteroid, stabilizes the thus obtained formulation, even when contained in an aluminum can, also with the possibility to exploit a synergic effect between the selected acids, as herein detailed.

According to one embodiment, the formulation of the invention is characterized by comprising a mixture of two or more monoprotic or polyprotic acids, preferably inorganic acids, said mixture at least containing hydrochloric acid (HCl) and/or phosphoric acid (H₃PO₄).

In one particularly preferred embodiment, the formulation of the invention comprises a mixture of HCl and H₃PO₄. In this respect, it has been surprisingly found that a formulation suitable for pMDI administration and comprising at least a LABA agent, and optionally a LAMA agent and/or a corticosteroid, is particularly stable when a combination of a selected molar ratio of HCl and H₃PO₄ is used. From the data collected in the herein below experimental part, it is evident that the use of the two acids improves the stability in a synergic way, with respect to H₃PO₄ alone. This effect not only provides an increase in the stability, but also endows the thus obtained formulation with a degree of stability in aluminum can, comparable to the one obtainable by using HCl alone with the FEP technology.

Thus, in one aspect, the formulation of the invention comprises a mixture of two inorganic acids, preferably, HCl and H₃PO₄ in a molar ratio, intended as moles of HCl/H₃PO₄, comprised from about 0.0018 and 0.0030, preferably from about 0.0020 and 0.0030. More preferably the molar ratio HCl/H₃PO₄ is comprised from about 0.0022 to 0.0028, still more preferably the molar ratio HCl/H₃PO₄ is comprised from about 0.0023 to 0.0027.

Advantageously, the preferred molar ratio can be set by properly dosing the acids when used in different concentrations, e.g. expressed as molarity or % w/w.

In one preferred embodiment, the HCl is 1M, i.e. a defined amount of an aqueous solution comprising 1M HCl is added to the pharmaceutical formulation. In another preferred embodiment the H₃PO₄ is added at concentration of 85% w/w, i.e. a defined amount of H₃PO₄ (85% by weight in water, based on the total weight of H₃PO₄ and water) is added to the pharmaceutical formulation.

According to the invention, the amount of 1M HCl contained in the pharmaceutical formulation is in a range from about 0.019 to 0.021% w/w (based on the total weight of the formulation) and the amount of H3PO4 85% w/w is in a range from about 0.001 to 0.002% w/w (based on the total weight of the formulation).

Preferably, the amount of HCl is in a range from about 0.019 to 0.021% w/w (based on the total weight of the formulation) and the amount of H3PO4 85% w/w is 0.001% w/w (based on the total weight of the formulation). More preferably, the amount of HCl is 0.019% w/w (based on the total weight of the formulation) and the amount of H₃PO₄ 85% w/w is 0.001% w/w (based on the total weight of the formulation).

As shown in the experimental part, Table 2, the addition of a mixture of HCl and H₃PO₄ to a formulation comprising formoterol fumarate, glycopyrronium bromide and BDP, contained in an aluminum can, increases the stability of the formulation in terms of % residue of the active ingredients, in particular formoterol fumarate, with respect to the corresponding formulations comprising the single acid, taken alone. As it can be appreciated said combination of inorganic acids, is in fact able to stabilize not only the formoterol fumarate, but also the other active ingredients contained in the formulation, such as the glycopyrronium bromide and the beclometasone dipropionate, to a such degree which is comparable with the stability obtained by using the FEP technology.

The present invention brings several advantages to the prior art, such as the increase of the stability of the formulation over the time, good shelf life, good reproducibility of the final formulation, the maintenance of optimal chemical conditions within cans readily available in commerce, and a consistent delivery and an efficacy of medication, particularly when formulated as a solution for a pMDI device.

Even further, the preferred combination of the selected acids, may also avoid the use of FEP coated can, thus providing a simpler manufacturing process and final device system. As known from the prior art and as above set forth, the formulation comprising formoterol and glycopyrronium bromide contained in a FEP coated can is in fact endowed with an improved stability, not achievable when the same formulation is contained e.g. in an aluminum can.

We have now found that the combination of inorganic acids, in particular the combination of HCl and H₃PO₄, is unexpectedly able to provide a degree of stabilization of a formulation according to the present invention, when contained in aluminum can, which is comparable with the stabilization degree obtained using the FEP technology of the prior art as can be observed in Tables 2 and 3.

According to the invention, the present formulation can be a solution, a suspension or a system comprising solution and suspension.

In a preferred embodiment, the formulation of the invention is a solution. Preferably one or more (more preferably all) of the pharmaceutically active components of the formulation, e.g. the LABA, LAMA and/or corticosteroid are completely and homogenously dissolved in the propellant and co-solvent.

Still more preferably, the formulation of the invention comprises a LABA agent, a mixture of at least two inorganic acids, preferably HCl and H₃PO₄, and/or a corticosteroid.

In one embodiment, the LABA agent of the formulation according to the invention, is selected from the group consisting of: fenoterol, formoterol fumarate, formoterol fumarate dihydrate, arformoterol, carmoterol (TA-2005), indacaterol, milveterol, bambuterol, clenbuterol, vilanterol, olodaterol, abediterol, terbutaline, salmeterol, diastereoisomeric mixtures, and a pharmaceutically acceptable salt thereof or hydrate thereof.

In one embodiment, the LABA is formoterol fumarate, preferably formoterol fumarate dihydrate.

In another embodiment, the formulation of the present invention comprises salbutamol, or (R)-salbutamol (levalbuterol) or a pharmaceutically acceptable salt thereof or hydrate thereof.

Preferably, the amount of LABA according to the present invention is comprised between 0.0005-0.04% w/w, more preferably between 0.001-0.03% w/w, even more preferably between 0.005-0.02% w/w.

In one embodiment, the LAMA agent of the formulation according to the invention, is selected from the group consisting of: glycopyrronium, ipratropium, oxitropium, trospium, tiotropium, aclidinium and umeclidinium with any pharmaceutically counterion thereof.

Preferred LAMA agent is glycopyrronium bromide.

In one embodiment, the LAMA agent, preferably glycopyrronium bromide, is present in the formulation of the invention in an amount in the range from 0.005 to 0.14% (w/w), preferably from 0.010 to 0.13% (w/w), more preferably from 0.010 to 0.045% (w/w), wherein % (w/w) means the amount by weight of the component, expressed as percent with respect to the total weight of the composition.

In one embodiment, the corticosteroid component of the formulation according to the invention, is selected from the group consisting of: budesonide, beclometasone, e.g. as the mono or the dipropionate ester, flunisolide, fluticasone, e.g. as the propionate or furoate ester, ciclesonide, mometasone, e.g. as the furoate ester, mometasone desonide, rofleponide, hydrocortisone, prednisone, prednisolone, methyl prednisolone, naflocort, deflazacort, halopredone acetate, fluocinolone acetonide, fluocinonide, clocortolone, tipredane, prednicarbate, alclometasone dipropionate, halometasone, rimexolone, deprodone propionate, triamcinolone, betamethasone, fludrocoritisone, desoxycorticosterone, rofleponide, etiprednol dicloacetate.

Beclometasone dipropionate (BDP) and budesonide are particularly preferred.

In a still preferred embodiment, the corticosteroid component is beclometasone dipropionate (BDP).

According to another embodiment of the present invention, the amount of the corticosteroid component, preferably BDP, is comprised between 0.01-0.7% w/w, more preferably between 0.05-0.5% w/w, even more preferably between 0.08-0.35% w/w.

In one embodiment, the present invention refers to a formulation, preferably a solution suitable for pMDI administration, comprising: a LABA agent, a LAMA agent, a corticosteroid and a mixture of at least two inorganic acids.

In a further preferred embodiment, the present invention refers to a formulation, preferably a solution suitable for pMDI administration, comprising: a LABA agent, a LAMA agent, a corticosteroid and a mixture of HCl and H3PO4.

In a still preferred embodiment, the present invention refers to a formulation, preferably a solution suitable for pMDI administration, comprising: a LABA agent, a LAMA agent, a corticosteroid and a mixture of HCl and H₃PO₄ in a molar ratio HCl/H₃PO₄ comprised from about 0.0018 to 0.0030, preferably from about 0.0020 to 0.0030, more preferably from about 0.0022 to 0.0028, even more preferably from about 0.0023 to 0.0027.

In a particularly preferred embodiment, the present invention refers to a formulation, preferably a solution suitable for pMDI administration, comprising: formoterol fumarate, glycopyrronium bromide, BDP and a mixture of at least two inorganic acids.

In a still preferred embodiment, the present invention refers to a formulation, preferably a solution, comprising: glycopyrronium, formoterol, BDP, and a mixture of HCl and H₃PO₄.

In a still preferred embodiment, the present invention refers to a formulation, preferably a solution, comprising: formoterol fumarate, glycopyrronium bromide, BDP, and a mixture of HCl and H₃PO₄ in a molar ratio HCl/H₃PO₄ comprised from about 0.0018 to 0.0030, preferably from about 0.0020 to 0.0030, more preferably from about 0.0022 to 0.0028, even more preferably from about 0.0023 to 0.0027.

As above indicated, the formulation of the invention is particularly suitable for the administration as a pMDI solution. In this respect, the present formulation also comprises a propellant and preferably, a co-solvent, as herein below described.

The propellant of the formulation according to the invention is selected from hydrofluoroalkane (HFA) and hydrofluoroolefins (HFOs) and a mixture thereof.

In one embodiment, the hydrofluoroalkane propellant is selected from the group consisting of: HFA134a (1,1,1,2-tetrafluoroethane), HFA 227 (1,1,1,2,3,3,3-heptafluoropropane, HFA152a (1,1-Difluoroethane) and mixtures thereof.

In one embodiment, the HFO propellant of the formulation according to the invention is selected from the group consisting of: 1,3,3,3-tetrafluoropropene (HFO-1234ze) and 2,3,3,3-tetrafluoropropene (HFO-1234yf).

Preferably the propellant is an HFA propellant, more preferably HFA134a.

In an equally preferred embodiment, the propellant is HFA152a.

HFAs or HFOs may be present in the formulation in an amount in the range from 75 to 95% (w/w), preferably from 85 to 90% (w/w), based on the total weight of the formulation.

As above set forth, in one embodiment the formulation comprising the mixture of inorganic acids according to the invention, may optionally further comprise additional components such as excipients, additives or low volatility components. The addition of said components may be suitably calibrated in order to modulate e.g. the chemical-physical properties of the formulation. In this respect, and also according to the above described preferred embodiments, the invention refers to a formulation as above described in detail, also comprising an HFA or HFO propellant, a co-solvent and optionally a low volatile component.

Preferably, said co-solvent is a polar compound able to increase the solubility of the components within the formulation. Preferred co-solvents are aliphatic alcohols having from 1 to 4 carbon atoms, such as methanol, ethanol, propanol, isopropanol and the like, preferably ethanol, more preferably anhydrous ethanol.

When present, said co-solvent is used in an amount comprised from 5% w/w and 20% w/w, more preferably from 10% and 15% w/w, based on the total weight of the formulation.

When present, the low volatility component is a compound characterized in having a vapor pressure at 25° C. lower than 0.1 kPa, preferably lower than 0.05 kPa. Preferred low volatility components are selected from the group consisting of: glycols, propylene glycol, polyethylene glycol, glycerol or esters thereof, ascorbyl palmitate and isopropyl myristate, wherein isopropyl myristate and glycerol are particularly preferred.

In a preferred embodiment, the present invention refers to a formulation, preferably a solution suitable for pMDI administration, comprising, consisting of or consisting essentially of: a LABA agent, a LAMA agent and/or a corticosteroid, a mixture of at least two inorganic acids, a propellant and an aliphatic alcohol having from 1 to 4 carbon atoms, preferably ethanol, more preferably anhydrous ethanol.

In a still preferred embodiment, the present invention refers to a formulation, preferably a solution suitable for pMDI administration, comprising, consisting of or consisting essentially of: a LABA agent, a LAMA agent and/or a corticosteroid, a mixture of HCl and H₃PO₄, an HFA propellant and an aliphatic alcohol having from 1 to 4 carbon atoms, preferably ethanol, more preferably anhydrous ethanol.

In a further preferred embodiment, the present invention refers to a formulation, preferably a solution suitable for pMDI administration, comprising, consisting of or consisting essentially of: glycopyrronium bromide, formoterol fumarate, BDP, a mixture of at least two inorganic acids, an HFA propellant and ethanol, more preferably anhydrous ethanol.

In a still preferred embodiment, the present invention refers to a formulation, preferably a solution suitable for pMDI administration, comprising, consisting of or consisting essentially of: glycopyrronium bromide, formoterol fumarate, BDP, a mixture of HCl and H₃PO₄, an HFA propellant, preferably HFA 134a or HFA 152a and ethanol, more preferably anhydrous ethanol.

In a further preferred embodiment, the present invention refers to a formulation, preferably a solution suitable for pMDI administration, comprising, consisting of or consisting essentially of: glycopyrronium bromide, formoterol fumarate, BDP, a mixture of HCl and H₃PO₄ in a molar ratio HCl/H₃PO₄ comprised from about 0.0018 to 0.0030, more preferably from about 0.0020 to 0.0030, more preferably from about 0.0022 to 0.0028, even more preferably from about 0.0023 to 0.0027, an HFA propellant, selected from HFA 134a and HFA 152a, and ethanol.

In a further preferred embodiment, the present invention refers to a formulation, preferably a solution suitable for pMDI administration, comprising, consisting of or consisting essentially of: glycopyrronium bromide, formoterol fumarate, BDP, a mixture of HCl and H₃PO₄ in a molar ratio between HCl and H₃PO₄ comprised from about 0.0022 and 0.0028, preferably from about 0.023 and 0.027, an HFA propellant selected from HFA 134a and HFA 152a, and ethanol.

In a further preferred embodiment, the present invention refers to a formulation, preferably a solution suitable for pMDI administration, comprising, consisting of or consisting essentially of: glycopyrronium bromide, formoterol fumarate, BDP, an amount of 1M HCl in a range from about 0.019 to 0.021% w/w (based on the total weight of the formulation), an amount of H₃PO₄ 85% w/w in a range from about 0.001 to 0.002% w/w (based on the total weight of the formulation), preferably 0.001% w/w (based on the total weight of the formulation), an HFA propellant selected from HFA 134a and HFA 152a, and ethanol.

In some embodiments, the formulation is free of further excipients other than those explicitly defined above. For instance, the formulation may be free of excipients other than the co-solvent, the propellant and two inorganic acids (e.g. HCl and H₃PO₄). Preferably the formulation is substantially free of further acids, more preferably substantially free of further acids or bases, other than those defined above (e.g. HCl and H₃PO₄).

As far as the can or canister is concerned, part or all of the canister of the pMDI device suitable to contain the formulation of the invention, may be made of a metal, e.g. aluminum, or metal alloys, stainless steel or anodized aluminum, fluorine passivated aluminum and the like. Alternatively, the canister may be a plastic can or a plastic-coated glass bottle.

The metal canisters may have part or all of the internal surfaces lined with an inert organic coating.

The coating is typically applied to the internal surface of the can, thus providing an internal layer acting as interface between the internal surface of the can, and the formulation therein contained.

In this regards, a suitable coated can of the invention may have part or all of its internal surfaces coated with an inert organic or inorganic coating such as for example fluorinated-ethylene-propylene polymer (FEP), polyether sulfone polymer (PES), a fluorinated-ethylene-propylene polyether sulfone polymer (FEP-PES), and the like, according to the prior art. However, an advantage of the present invention is that such coatings may not be necessary in order to achieve suitable stability, i.e. very high stability may achieved even in non-FEP-coated cans (e.g. standard aluminium cans).

In a preferred embodiment, the invention refers to the above described formulation, contained in a pMDI canister made of aluminum or stainless steel. Thus, in one aspect, the invention refers to a pMDI canister made of aluminum or stainless steel, filled with the formulation of the invention as above described in detail. Aluminum cans are preferred.

The canister of a pMDI device is typically crimped with a metering valve for delivering a therapeutically effective dose of the active ingredients. The metering valve assembly comprises at least one rubber gasket seal made of a proper elastomeric material selected from: EPDM (a polymer of ethylene-propylene-diene monomer), butyl or halo butyl rubbers such as chlorobutyl or bromubutyl rubbers (optionally halogenated copolymers of isobutylene with isoprene) TPE (thermoplastic elastomer), cycloolefin copolymer (COC) or combination thereof.

Suitable valves for the present invention are available on the market, e.g. from manufactures well known in the field, such as the Bespak, Aptar-Valois and V.A.R.I.

The metering valve according to the invention is typically capable of delivering a volume in the range from 25 to 150 μl, preferably in the range from 50 to 100 μl, and more preferably from 50 μl to 70 μl per actuation; the most preferred are 50, 63 and 100 μl per actuation.

The efficacy of a pMDI device is a function of the dose deposited at the appropriate site in the lungs. Deposition is affected by the aerodynamic particle size distribution of the formulation which may be characterised in vitro through several parameters.

The following parameters of the particles emitted by a pressurized pMDI may be determined:
i) mass median aerodynamic diameter (MMAD) is the diameter around which the mass aerodynamic diameters of the emitted particles are distributed equally;
ii) delivered dose is calculated from the cumulative deposition in the ACI, divided by the number of actuations per experiment;
iii) respirable dose (fine particle dose=FPD) is obtained from the deposition from Stages 3 (S3) to filter (AF) of the ACI, corresponding to particles of diameter<4.7 microns, divided by the number of actuations per experiment;
iv) respirable fraction (fine particle fraction=FPF) which is the percent ratio between the respirable dose and the delivered dose.
v) “extrafine” dose is obtained from the deposition from Stages 6 (S6) to filter, corresponding to particles of diameter≤1.1 microns, divided by the number of actuations per experiment.

According to a further aspect of the invention there is provided a method of filling an aerosol inhaler with a pharmaceutical composition of the invention. Conventional bulk manufacturing methods and machinery well known to those skilled in the art of pharmaceutical aerosol manufacture may be employed for the preparation of large-scale batches for the commercial production of filled canisters.

As a general example said methodology may comprise the steps of:
a) preparing a solution comprising: formoterol fumarate, BDP, glycopyrronium bromide and ethanol;
b) filling a canister with said solution;
c) adding an amount of HCl and H₃PO₄ resulting in molar ratio HCl/H₃PO₄ comprised from about 0.0018 to 0.0030;
d) crimping with a valve and gassing with HFA propellant.

The packaged formulations of the invention are stable for extended periods of time when stored under normal conditions of temperature and humidity.

Stability is assessed by measuring content of residual active ingredient.

In a further aspect, the invention refers to the above described formulation for use as a medicament. Thus, the invention refers to the use of the formulation as herein described for the preparation of a medicament.

Preferably, the formulation of the invention is for prophylactic purposes or for symptomatic relief of a wide range of respiratory disorders, such as asthma of all types and chronic obstructive pulmonary disease (COPD).

In one preferred embodiment, the invention refers to the formulation as herein described, for the treatment and/or prophylaxis of respiratory disorders, preferably for the treatment and/or prophylaxis of asthma or COPD.

Other respiratory disorders for which use of the pharmaceutical compositions of the invention may be beneficial are those characterized by obstruction of the peripheral airways as a result of inflammation and presence of mucus, such as chronic obstructive bronchiolitis, chronic bronchitis, emphysema, acute lung injury (ALI), cystic fibrosis, rhinitis, and adult or acute respiratory distress syndrome (ARDS).

As it is will be recognized, all the herein described embodiments are to be intended as included in the scope of the present invention, also in any possible combination with all the other preferred embodiments, as herein above and below set forth.

The invention will be now described by the following not limiting examples.

### EXPERIMENTAL PART

### Example 1

A study was performed to investigate the chemical stability of formulation intended for pMDI administration comprising formoterol fumarate dihydrate (FF), glycopyrronium bromide (GB) and beclometasone dipropionate (BDP). Said formulation is a solution, contained in aluminum can crimped with a Bespak valve having a 63 μl metering volume.

A different type and amount of acids either alone or in mixture thereof were added to the formulation, thus providing Formulations 1-4, as reported in Table 1 and 2.

TABLE 1

| | Formulation 1 | Formulation 2 | Formulation 3 | Formulation 4 |
|---|---|---|---|---|
| COMPONENT | % w/w | % w/w | % w/w | % w/w |
| FF | 0.008 | 0.008 | 0.008 | 0.008 |
| BDP | 0.136 | 0.136 | 0.136 | 0.136 |
| GB | 0.034 | 0.034 | 0.034 | 0.034 |
| 1M HCI | 0.019 | 0.021 | 0 | 0 |
| H₃PO₄ (85% w/w) | 0.001 | 0.001 | 0.001 | 0.002 |
| Ethanol anhydrous | 12 | 12 | 12 | 12 |
| HFA 134a | 87.8 | 87.8 | 87.8 | 87.8 |

The Formulations 1-4 were put in stability chambers at 40 C°, 75% R.H. in inverted position for 1 month (1M) and then check for API assay and relevant degradation products. APIs residue % are reported in Table 2.

TABLE 2

| | | 85% w/w | | | | |
|---|---|---|---|---|---|---|
| Formu- | 1M HCl | H₃PO₄ | Molar ratio | | | |
| lation | % w/w | % w/w | HCl/H₃PO₄ | FF % | GB % | BDP % |
| 1 | 0.019 | 0.001 | 0.0025 | 97.0 | 99.6 | 99.8 |
| 2 | 0.021 | 0.001 | 0.0028 | 96.1 | 100.8 | 100.9 |
| 3 | 0 | 0.001 | | 85.0 | 95.5 | 99.2 |
| 4 | 0 | 0.002 | | 86.0 | 98.3 | 99.9 |

As it can be observed by Table 2 when a mixture of HCl and H₃PO₄ is added according to Formulations 1-2, a significant improvement of the chemical stability of formoterol (FF), glycopyrronium bromide (GB) and beclometasone dipropionate (BDP) is achieved. Of note, the % FF can reach values even higher than 95%. In fact, the formulations 1 and 2 show a significantly improved stability, in terms of FF %, GB % and BDP % residue, for example when compared to the stability of the formulation 3 and 4 wherein the H₃PO₄ is present alone, and where the % FF is actually lower than 90%.

### Example 2

The same analysis of Example 1 has been ran using a correspondent formulation but in the presence of HCl only, and in an aluminum FEP coated can crimped with a Bespak valve having a 63 μl metering volume.

The thus obtained formulation (Form. FEP) was put in stability chambers at 40 C°, 75% R.H. in inverted position for 1 month (1M) and then check for API assay and relevant degradation products. API % residue and relevant total degradation products are reported in Table 3.

TABLE 3

| | 1M | 85% w/w | | | |
|---|---|---|---|---|---|
| | HCI % | H₃PO₄ | | | |
| | w/w | % w/w | FF % | GB % | BDP % |
| Form. FEP | 0.019 | 0.0 | 97.4 | 100.2 | 101.4 |

As evident from the comparison of the above Tables 2 and 3, the mixture of inorganic acids according to the invention provides a stabilization, in terms of residue % of the APIs, particularly regarding the formoterol, comparable to the high stabilization degree obtainable using the FEP technology. In both cases in fact the % FF can be even higher that 95%, thus representing a significant degree of stability.

1. A pharmaceutical composition, comprising:
a LABA agent;
a co-solvent;
a propellant; and
a mixture of at least two inorganic acids.

2. The pharmaceutical composition according to claim 1 wherein the LABA agent is at least one selected from the group consisting of fenoterol, formoterol fumarate, formoterol fumarate dihydrate, arformoterol, carmoterol (TA-2005), indacaterol, milveterol, bambuterol, clenbuterol, vilanterol, olodaterol, abediterol, terbutaline, and salmeterol, or a diastereoisomeric mixture thereof, or a pharmaceutically acceptable salt thereof, or a hydrate thereof.

3. The pharmaceutical composition according to claim 1, wherein the LABA agent is formoterol fumarate dihydrate.

4. The pharmaceutical composition according to claim 1, wherein the mixture of at least two inorganic acids comprises at least HCl.

5. The pharmaceutical composition according to claim 1, wherein the mixture of at least two inorganic acids comprises at least H₃PO₄.

6. The pharmaceutical composition according to claim 1, wherein the mixture of at least two inorganic acids is a mixture of HCl and H₃PO₄.

7. The pharmaceutical composition according to claim 6, wherein a molar ratio of HCl to H₃PO₄ in the composition is 0.0018 to 0.0030.

8. The pharmaceutical composition according to claim 7, wherein the molar ratio of HCl to H₃PO₄ is 0.0022 to 0.0028.

9. The pharmaceutical composition according to claim 7, wherein the molar ratio of HCl to H₃PO₄ is 0.0023 to 0.0027.

10. The pharmaceutical composition according to claim 1, comprising:
1M HCl in an amount of from about 0.019 to 0.021% w/w based on a total weight of the formulation; and
H₃PO₄ 85% w/w in an amount of from about 0.001 to 0.002% w/w based on the total weight of the formulation.

11. The pharmaceutical composition according to claim 10, comprising:
1M HCl in an amount of from about 0.019 to 0.021% w/w based on the total weight of the formulation; and
H₃PO₄ 85% w/w in an amount of 0.001% w/w based on the total weight of the formulation.

12. The pharmaceutical composition according to claim 1, further comprising at least one LAMA agent selected from the group consisting of glycopyrronium, ipratropium, oxitropium, trospium, tiotropium, aclidinium, and umeclidinium, with any pharmaceutically acceptable counterion thereof.

13. The pharmaceutical composition according to claim 12, wherein the LAMA agent is glycopyrronium bromide.

14. The pharmaceutical composition according to claim 1, further comprising at least one corticosteroid selected from the group consisting of: budesonide, beclometasone (BDP), flunisolide, fluticasone, ciclesonide, mometasone, mometasone desonide, rofleponide, hydrocortisone, prednisone, prednisolone, methyl prednisolone, naflocort, deflazacort, halopredone acetate, fluocinolone acetonide, fluocinonide, clocortolone, tipredane, prednicarbate, alclometasone dipropionate, halometasone, rimexolone, deprodone propionate, triamcinolone, betamethasone, fludrocoritisone, desoxycorticosterone, rofleponide, and etiprednol dicloacetate.

15. The pharmaceutical composition according to claim 14, wherein the corticosteroid is budesonide or beclometasone dipropionate (BDP).

16. The pharmaceutical composition according to claim 15, wherein the corticosteroid is beclometasone dipropionate (BDP).

17. The pharmaceutical composition according to claim 1, wherein the composition is a solution.

18. The pharmaceutical composition according to claim 1, wherein the co-solvent is an aliphatic alcohol having from 1 to 4 carbon atoms.

19. The pharmaceutical composition according to claim 18, wherein the co-solvent is ethanol.

20. The pharmaceutical composition according to claim 1, wherein the propellant is at least one selected from the group consisting of a hydrofluoroalkane (HFA) and a hydrofluoroolefin (HFO).

21. The pharmaceutical composition according to claim 20, wherein the propellant is at least one selected from the group consisting of HFA134a and HFA152a.

22. A canister, containing the pharmaceutical composition according to claim 1, wherein the canister is made of at least one selected from the group consisting aluminum, stainless steel, anodized aluminum, and fluorine passivated aluminum.

23. A canister configured for use in a pMDI device, containing the pharmaceutical composition according to claim 1.

24. The canister according to claim 23, made of aluminum.

25. (canceled)

26. A method for treating a respiratory disorder, comprising administering the pharmaceutical composition according to claim 1 to a subject in need of such treatment.

27. A method for treating asthma or COPD, comprising administering the pharmaceutical composition according to claim 1 to a subject in need of such treatment.
